(19) United States
(12) Patent Application Publication
(10) Pub. No.: US 2011/0197649 A1
(43) Pub. Date: Aug. 18, 2011

(54) SELF-CALIBRATING GAS SENSOR

(75) Inventors: Jie HAN, Wuxi (CN); Lijun SHEN, Wuxi (CN); Lei XIE, Wuxi (CN)

(73) Assignee: WUXI SUNVOU BIOTECH CO., LTD., Wuxi (CN)

(21) Appl. No.: 13/042,747

(22) Filed: Mar. 8, 2011

Related U.S. Application Data
(63) Continuation of application No. PCT/CN2008/073387, filed on Dec. 9, 2008.

(30) Foreign Application Priority Data
Sep. 8, 2008 (CN) 200810196517.7

(51) Int. Cl. G01N 33/00

(52) U.S. Cl. 73/1.06

(57) ABSTRACT

A self-calibrating gas sensor comprises a steady current measuring gas line composed of valves, pumps and a current-type electrochemical sensor, and a coulomb analyzing gas line composed of said valves, said pumps, said current-type electrochemical sensor and a sample chamber. The two gas lines can be interchanged between measurement and analysis by controlling valves. The sensor can measure gas concentrations, and self-calibrate its sensitivity without the need of standard gases for external calibration.

### FIELD OF THE INVENTION

The present invention relates to gas sensor field, in particular to a gas sensor that has self-calibration function or does not require calibration.

### BACKGROUND OF THE INVENTION

To ensure the reliability and accuracy of gas sensors, the gas sensors must be calibrated with cylinder gas at known concentration to calibrate sensitivity and set zero point for the gas sensors when the gas sensors are shipped from the factory or during the operation process of the gas sensors. The user can calibrate the instruments with the method and procedure recommended by the manufacturer or returns the instruments to the manufacturer or a designated service agency for calibration. Frequency and specialized requirements for calibration bring a great deal of inconvenience to both the manufacturer and the user, and increase the operation cost and use cost. Therefore, the calibration of gas sensors is always a great concern for manufacturers and users.

At present, a main method for solving that problem is to provide safe, convenient, and reliable portable automatic calibrators. For example, Chinese Patent Application No. 1221804C disclosed such an instrument. This instrument integrates with a small gas cylinder and a flow controller to provide a portable calibrating device for gas detectors or sensors, so that the user can use the instrument to calibrate the gas detectors or sensors at any time. In addition, US Patent Application No. U.S. Pat. No. 2,554,153 disclosed an automatic calibration apparatus for gas sensor, and the apparatus uses gas cylinder at two different concentrations to calibrate the sensors. A method that utilizes integral electrolysis for external calibration and does not require known gas concentration is described in US Patent No. U.S. Pat. No. 4,829,809. In that invention, the electrochemical sensor to be calibrated is placed in a chamber of known volume, and the sensor sensitivity and gas concentration are calculated with a curve of sensor current versus time. However, this method still belongs to an external calibration method, and the gas used in the calibration is still simulated gas rather than the gas to be actually detected.

At present, all efforts only focus on miniaturization and automation of lab calibration methods. With such calibration methods, frequent external specialized calibrations are still required; in addition, whether these calibration methods can ensure reliability and accuracy of detection is still a problem, for the calibration conditions (carrier gas, temperature, pressure, gas flow condition, and humidity, etc.) usually can not fully reflect or simulate the actual working conditions of the gas sensors.

An important characteristic of the present invention is: the sensor is calibrated with a sample gas containing the gas to be detected under actual detection conditions, instead of an external calibration under simulated conditions. Therefore, there is no error caused by the difference between simulated conditions and actual conditions. The present invention not only eliminates the operation cost and use cost incurred by calibration for the manufacturer and the user, but also improves the operational reliability of sensors.

### SUMMARY OF THE INVENTION

To overcome the drawbacks in the prior art, including the inventions described above, the present invention provides a self-calibrating gas sensor that does not require external calibration.

The present invention relates to a calibration which is a self-calibration of sensor with the sample gas containing the gas to be detected under the actual detection conditions. As an embodiment and application example described below, this self-calibration is based on coulomb analysis in electrochemical analysis. controlled potential electrolysis is a common analytical method in electrochemical analysis. For gas analysis, if the electrolytic efficiency of the tested gas is 100%, the Faraday Law applies when the active components in a fixed system is electrolyzed.

Q=∫Idt=nFNo (1)

Wherein, No is the total amount of active components in the system, Q is the total electricity consumption, F is Faraday constant, I is electrolytic current, and t is time.

Since current I and time t can be measured accurately, if the volume of the system is determined, the concentration of the active components can be calculated directly from Q.

For a reaction system under specific conditions, if the reaction current of the active components on the electrodes of the electrochemical sensor is directly proportional to the gas concentration at any time (this condition can be met in first-order reactions or mass transfer controlled reactions, and applies to most electro-chemical gas sensors), the response current of the sensor to the gas sample meets the following equation:

I(t)=I(0)EXP(−pt)=nFkCo(t) (2)

Wherein, I(0) is the initial current, and p, k are constants.

Therefore, in a controlled potential integral electrolytic process, the concentration and current attenuate exponentially with time, and ultimately reach the background current level.

The following equation can be obtained from equation (2):

Ln(I(t))=ln(I(0))−kt (3)

The intercept ln(I(0)) and slope k can be calculated with graphing method (ln(I(t)) vs. t) or with regression analysis.

The following equation can be obtained by integration of equation (1):

Q=∫Idt=∫I(t)EXP(−pt)dt=I(0)*/2.303k*(1−10^{-kt}) (4)

When t is high enough:

Q=I(0)*/2.303k (5)

Thus, the concentration can be determined as follows:

C(0)=N/V=Q/nFV=I(0)/2.303knFV (6)

Therefore, if a cyclic electrolysis gas circuit that comprises this gas sensor can be added in the measurement gas circuit of the gas sensor, the unknown concentration of the gas sample can be determined by using above analytical result, and thereby detection without calibration or self-calibration without external operation can be achieved. To this end, on the basis of the steady-state detection gas circuit composed of gas-sensitive element and sampling device in a gas sensor, a cyclic calibration gas circuit for self-calibration, which is composed of the gas sensor, an gas pump, and a sample chamber, is provided by the present invention, and the switching between detection mode and calibration mode is controlled by means of a control valve, to implement two functions (i.e., detection and calibration) with the same sensor and the same gas.

Compared to all existing gas sensors, which require frequent external calibrations, the present invention has great advantages. The present invention not only eliminates all investment and maintenance cost required for external calibration, but also avoids potential safety hazards and many other inconvenient factors related with external calibration. More particular, since the calibration in the present invention is implemented under actual detection conditions instead of external simulated conditions, the detection reliability is ensured.

The above and other characteristics, features, and advantages of the present invention will be understood more clearly in the following description of embodiments, with reference to the accompanying drawings.

### DESCRIPTION OF DRAWINGS

Hereunder the present invention will be explained in greater detail with the description of the embodiments and claims, with reference to the accompanying drawings. In the drawings, same reference numbers refer to same characteristics, wherein:

FIG. 1 is a structural diagram of the self-calibrating gas sensor provided in the present invention;

FIG. 2 is a calibration curve of the self-calibrating gas sensor provided in the present invention;

FIG. 3 is another combination of the gas circuits in the sensor module of the present invention.

### DETAILED DESCRIPTION OF THE EMBODIMENTS

FIG. 1 is a structural diagram of the self-calibrating gas sensor module provided in the present invention. This sensor module comprises a gas inlet valve 1, a sample chamber 2, a gas pump 3, a sensor 4, a gas outlet valve 5, and a valve 6.

First, the gas sample that carries the gas to be detected is exhausted from inlet 1 through the steady-state detection gas circuit arranged in 1-2-3-4-5 sequence. The gas circuit is usually used for detection. When calibration is required, the purpose of this procedure is to replace or remove the original existing gas in the gas circuit, and measure the steady-state response current value Ic of the gas sensor at the concentration. After the steady-state response current value is obtained, the gas inlet value 1 and gas outlet value 5 are closed while the valve 6 is open at the same time, to start the cyclic calibration process. At this point, the volume of the gas sample is the volume of the gas circuit.

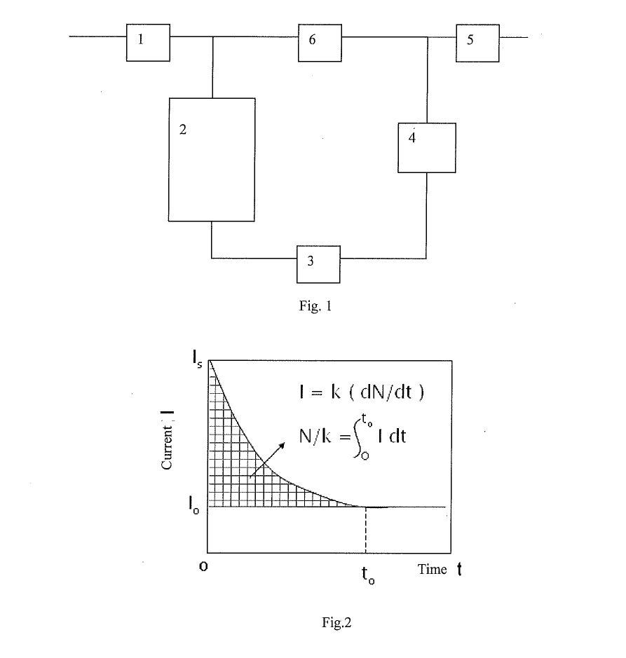

The calibration is carried out in a gas circulation process driven by the gas pump in the circulation gas circuit for calibration arranged in 2-3-4-6-2 sequence, and repeated the circulation until the detection current attenuates to 10% of the initial value or lower. Thus, the curve of detection current versus time illustrated in FIG. 2 is obtained. Then, the concentration C(0) of gas sample is obtained from equations (3), (5), and (6), and then the sensitivity of the sensor is calibrated by Lc/C(0).

It must be noted that the core of the present invention is the cyclic calibration gas circuit composed of gas-sensitive element, gas pump, sample chamber, and control valve, etc. These parts can also be arranged in other combination to form other structures, in addition to the structure shown in FIG. 1.

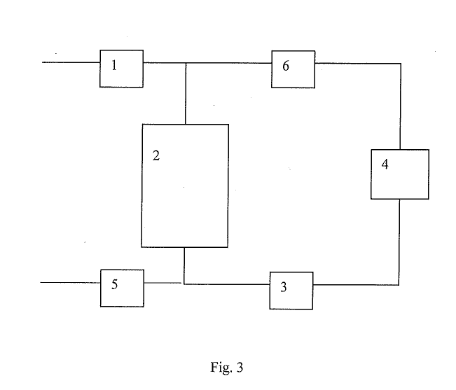

For example, the air outlet valve 5 shown in FIG. 1 can be arranged after the sample chamber (as shown in FIG. 3). To start measurement, the gas in the sample chamber is displaced first (feed gas directly by means of the pump or an external gas source, such as exhalation), and then the valves 1 and 5 are closed, while value 6 is opened at the same time to form a cycle analysis gas circuit, so as to carry out electrolytic analysis for the gas in the sample chamber.

For the application shown in FIG. 1, it is preferred that the gas in the sample chamber can form piston gas flow under the driving of gas pump 3, so that the gas in the sample chamber can be refreshed fully and quickly. For the application shown in FIG. 3, it preferred that the pipe diameter of the sample chamber should be large enough, in order to ensure the gas in the sample chamber can be refreshed quickly and completely at the sampling flow rate (a relatively high flow rate).

As another structure of the present invention, the sample chamber can be in the form of a moving piston which is similar to a syringe. or a sampling gas bag In such a structure, the gas in the gas bag or syringe is exhausted first, and then the sample gas is filled to carry out analysis.

As another structure of the present invention, the sample chamber can be an enclosed container, filled with clean air initially. To start analysis, gas of certain volume can be filled into the container.

The same principle and method also apply to other types of gas-sensitive elements, such as oxide semiconductor gas-sensitive elements and catalytic combustion gas-sensitive elements, etc. For these gas-sensitive elements, the cyclic calibration gas circuit described in the present invention can be used. First, the detected gas is consumed completely; then, the concentration of the detected gas can be determined from the consumed quantity under the mass action law, and thereby self-calibration can be carried out.

All these fall in the protection scope of the present invention.

### Embodiment 1

This embodiment is provided to illustrate how to calibrate a hydrogen sulfide gas sensor with unknown sensitivity without any specialized external calibration by the present invention. The sensitivity of the sensor has drifting due to the effects of humidity and other interfering gas in the working environment. When such a sensor is used for exhalation detection against oral diseases, its sensitivity has to be calibrated frequently in view of the requirements for high sensitivity and accuracy. In contrast, when such a sensor is used for gas detection in an industry or environment without strict sensitivity and accuracy requirements, the calibration usually is not so frequent. Generally, the sensor should be returned to the manufacturer for calibration or calibrated by the user through an external calibration process with the cylinder gas and method provided by the manufacturer.

The testing device in this embodiment is shown in FIG. 1. For the convenience of test verification, first, standard hydrogen sulfide gas at 30 ppm concentration is pumped by pump 3 into the gas circuit arranged in 1-2-3-4-5 sequence to carry out measurement, until the steady-state current Ic (24.2 uA in this embodiment) is obtained; then, valves 1 and 5 are closed, while the gas pump and valve 6 are opened at the same time to start cyclic electrolysis, in order to obtain the current attenuation curve; next, the gas concentration is calculated with the volume of sample chamber (5.2 mL) as 30.1 ppm; thereby, the sensitivity of the sensor is calculated as 0.80 uA/ppm. The error between the self-calibration value and the standard gas concentration is only 0.33%, which is within the range of sensor error.

### Embodiment 2

This embodiment is provided to illustrate how the present invention is used for self-calibration of an expiratory gas nitrogen oxide sensor. As an indicator of respiratory inflammation, expiratory gas nitrogen oxide can be used to diagnose and track respiratory diseases such asthma. In European and American countries, standards are established to encourage and recommend the application of such non-intrusive diagnostic techniques, and specify the minimum detection accuracy should not be higher than 5 ppb. For detection at such low concentration, the sensitivity of gas sensor may drift quickly and severely due to the effects of ambient humidity and other interfering gasses. Specialized calibrations have to be carried out more frequently than the case of detection at higher concentration.

For example, the Patent Application US20040082872, detection and analysis for expiratory gases at high sensitivity is implemented by strictly controlling the temperature (22° C.) and humidity (70%) of the sample gas and the temperature (22° C.) of the gas sensor, and sensitivity drift affected by temperature and humidity is reduced to some degree. However, the sensitivity of sensor may still drift quickly and severely after the sensor is used for many times or due to the effects of other interfering gasses or aging or passivation of the detection electrodes. Thus, the sensor has to be replaced or calibrated regularly; for example, the sensor has to be calibrated by a specialized technician through an external calibration process with the method provided by the manufacturer once in every 7 days or after the sensor is used for certain times.

Such frequent external calibration is not required if the present invention is applied. Instead, all calibrations can be carried out on the sensor internally. The testing device in this embodiment is shown in FIG. 3. During the measurement, nitrogen oxide gas at 40 ppb is prepared from standard gas, and filled into sample chamber 2 to replace the gas in the sample chamber completely; then, the valves 1 and 5 are closed, while the gas pump and the valve 6 are opened to carry out cyclic electrolysis; next, the concentration of nitrogen oxide can be calculated with the volume of sample chamber (136 mL) and the sensor current attenuation curve. This embodiment is verified for three times, and the concentration values obtained during the self-calibration are 41.5, 41.7, and 41.5 ppb respectively. The error between these calibration values and the standard gas concentration 40 ppb is within the specified accuracy range of the sensor, demonstrating the reliability of the self-calibration method provided in the present invention.

The above embodiments are provided only for those skilled in the art to implement or use the present invention. Those skilled in the art can make modifications or variations to the above embodiment, without departing from the spirit of the present invention. Therefore, the protection scope is not limited to the above embodiments, but should be the maximum scope of the innovative characteristics as described in the claims.

1. A self-calibrating gas sensor, wherein, comprising:
a valve, which controls the direction of gas flow; preferably a solenoid electric valve;
a gas pump, which is used to transfer gas; preferably a steady-flow circulation gas pump;
a gas-sensitive element, with current response in directly proportional to the concentration of the detected gas; preferably a current-type electrochemical sensor;
a sample chamber, which is used to store the gas sample.

2. The self-calibrating gas sensor according to claim 1, wherein:
The valve, gas pump, and gas-sensitive element form a gas circuit for steady state measurement, designed for concentration measurement;
The valve, gas pump, gas-sensitive element, and sample chamber form a calibration circulation gas circuit, designed for self-calibration.

3. The self-calibrating gas sensor according to claim 1, wherein:
when the gas circuit for steady state measurement is used for concentration measurement, the gas pump intakes the gas to be analyzed into the sensor at a constant flow rate, wherein, some gas is stored in the sample chamber, while the remaining gas is exhausted through the outlet;
when the calibration circulation gas circuit is used for self-calibration, the gas pump feeds the gas in the sample chamber at a constant flow rate into the gas-sensitive element for electrolysis and then returns the gas to the gas chamber; the above process is repeated the until the gas is completely electrolyzed and at the same time, the current change versue time is logged and thereby the gas concentration is calculated.

4. The self-calibrating gas sensor according to claim 1, wherein:
the volume of the sample chamber accounts for more than 99% of the total volume of the circulation gas circuit.
